(19) United States
(12) Patent Application Publication
(10) Pub. No.: US 2006/0263455 A1
(43) Pub. Date: Nov. 23, 2006

(54) Composition for preventing urinary system infections

(75) Inventor: Jean-Christophe Anton, Strasbourg (FR)

Correspondence Address: YOUNG & THOMPSON, 745 SOUTH 23RD STREET, 2ND FLOOR, ARLINGTON, VA, 22202, US

(73) Assignee: NATURAL PRODUCT CONSULTING, Strasbourg (FR)

(21) Appl. No.: 10/558,744

(22) PCT Filed: Jun. 18, 2004
(86) PCT No.: PCT/FR04/01523

§ 371 Date: Dec. 1, 2005

(30) Foreign Application Priority Data
Jun. 20, 2003 (FR) 0307465

(51) Int. Cl. A61K 36/8962; A61K 36/87; A61K 36/45; A61K 31/70; A61K 31/202

(52) U.S. Cl. 424/732; 424/754; 424/766; 514/23; 514/560

(57) ABSTRACT

Compositions for preventing urinary system infections, contain constituents provided for exerting simultaneous actions in order to suppress pathogenic germs causing these infections or to reduce their harmfulness and to reinforce the integrity of the wall of urinary paths. The first action is obtained by constituents which exert an antibacterial action onto germs present in the urinary paths, an antibacterial action onto germs present in the gastro-intestinal tract thereby reducing the colonization by these urinary path germs, and reduce their ability to adhere to the wall of the urinary system. The constituents exerting the first action are polyphenols extracted from garlic and from plants selected from: Ericaceae, Saxifragaceae, Vitaceae and Caprifoliaceae families. The second action is obtained by polyunsaturated fatty acids of the family n-3 that enable the reduction of wall inflammation and the bringing of the immune system back into balance.

The present invention relates to compositions for the prevention of infections of the urinary system, which compositions are accordingly essentially used prophylatically.

Urinary infections comprise on the one hand the asymptotic microbial colonization of the urine, and on the other hand the symptomatic infection with microbial invasion and inflammation, of the structures of the urinary tree. Bacteria are by far the microorganisms that most frequently infect, but fungi and viruses can also produce urinary infection. The microorganisms proliferate and the inflammatory cells present in the urine constitute the biological characteristic of this affliction.

Urinary infections are one of the most frequent pathologies encountered by doctors, the great majority in women, and particularly in the form of cysts or inflammation of the bladder. These latter are recurrent infections, which can be frequent. In this case, the available antibiotic treatments are effective, but often poorly tolerated, and they moreover give rise to increasing resistance on the part of the uropathogenic germs. As to the increase in the power of resistance to antibiotics, the medical field is encouraging doctors to question more systematically the appropriateness of the use of antibiotics.

In many cases, those having recurrent urinary infections suffer from a large decline in the quality of their life and extreme fatigue can lead to depression. The appearance in recent years of resistance to antibiotics leads to increase the frequency of relapse, and further decreases the quality of life of the patient whilst increasing the economical cost of these pathologies for society. For all these reasons, spacing apart recurrences, or in other words avoiding relapses, becomes a priority objective both for persons suffering from these pathologies and for the scientific community.

The principal means of natural defense against these urinary infections relate to the volume and dynamics of the urinary flow (apart 1.5 liter per day), permitting regular and complete emptying of the bladder (4 to 5 times per day), as well as the antibacterial properties of the epithelium bordering the urinary apparatus (urothelium). The integrity and impermeability of the urothelium which covers the urinary cavities is thus particularly important. Among other means, could be cited the secretion of a particular protein (Tamm-Horsfall) secreted by the liver and present in the urine, as well as the vaginal secretions of women and prosthetic secretions of men.

In general, the treatment presently recommended for a recurring urinary infection does not differ from that for a single episode. For persons having more than three infections per year, certain prophylactic treatments based on medications of the antibiotic type are provided. But apart from hygienic measures and treatment over several years with moderate doses of antibiotics, there is at present no preventive treatment that is satisfactory over the long term, for urinary infections, and particularly cystitis.

Given the limitations mentioned, which are seen to arise from antibiotic therapies, the preventive treatment of urinary infections must necessarily be found in new ways.

Patent application WO 97/29763 thus proposes compositions seeking to treat urogenital and intestinal troubles, which compositions contain an effective quantity of at least one plant of the family Ericaceae or an extract of said plant and an effective quantity of a growth factor specifically stimulating the growth of acido-lactic bacteria.

The present invention proposes another solution, by using together active ingredients permitting preventing the recurrence of urinary infections. The combination of these active ingredients permits a preventive action which is more effective than the same action of a single one of the components taken separately, because they interact.

More precisely, the composition for the prevention of infections of the urinary system according to the invention is characterized in that it contains components provided to exert simultaneous actions for the purpose of:
suppressing pathogenic germs at the outset of cases of infection, or reducing their noxiousness;
reinforcing the integrity of the urinary wall.

In other words, the role of these active ingredients is to act simultaneously for the same objective (to prevent re-infections) but by different paths of approach: a direct action on the germs, and an indirect action on the wall of the urinary system.

As to the first action, the composition of the invention comprises, for the suppression and/or reduction of the noxiousness of pathogenic germs, components aimed at exerting an antibacterial action directly on the germs present in the urinary pathways, but also on the germs present in the gastrointestinal tract. By this latter action, the components aim to reduce the possibilities of colonization of the urinary pathways by germs from the digestive tract and thus limit the infections. The composition of the invention also comprises components to decrease the aptitude of the germs to adhere to the walls of the urinary system.

In the stage of adhesion to the urinary pathways by the infecting pathogenic germs, in most of the cases Escherichia coli, the bacterial factors which influence the initial adherence between the germ and the urogenital mucosa imply the expression of pili (such as P-fimbriae, localized on the membrane of Escherichia coli) which are over-represented in isolates of germs of patients suffering from cystitis. The adherence also implies the interaction between these pili and the uroplakin I located on the wall of the bladder. It is this mechanism which is the promoter of the adhesion and which is to be decreased.

From a viewpoint of reinforcing the integrity of the wall of the urinary system, components of the invention permit both decreasing inflammation of the wall, and rebalancing its immune system.

The integrity of the epithelium lining this wall is one of the key elements permitting the organism to fight infection. Thus, Escherichia coli is incapable of fixing itself to the intact mucosa of the walls of the urinary system. Instead, damage at the level of these mucosa permit the germs an access to the receptors present in the subjacent urothelium, and thus lead to the adherence of the germs on these walls.

To defend against this, the epithelium is capable of generating an exfoliation permitting it to shed germs adhering to the cells. To do this, it is necessary first of all to decrease the inflammation of the wall, an infected wall not being able to be used under good conditions these defense means in the presence of damage to the epithelium.

Damage is also produced because of disturbances of the immune system, which accordingly promote also the colonization of the epithelium by pathogenic germs.

In this case, the mastocytes, immune cells largely implied in cystitis in particular, produce alterations of the integrity of the mucosa by the process of inflammation. This is the reason why the regulation of the immune system and the decrease of information work hand in hand for the reinforcement of the integrity of the wall.

More precisely, according to the invention, the antibacterial agents lead to a suppression of the pathogenic germs or in a reduction of the noxiousness and are selected from the group of sulfurated compounds of the type allicin and derivatives and/or of the type of polyphenols.

Allicin has thus demonstrated antibacterial, antifungal and antiviral properties on strains of pathogenic agents often resistant to conventional therapies, which is to say based on antibiotics. Experimentally, the sensitivity of different preparations of bacteria and of virus to this substance, is all the more very significant.

Moreover, there is no phenomenon of resistance to allicin because its mode of action is completely different from that of antibiotics.

In the article of D. L. LAMM and D. R. RIGGS, Enhanced Immunocompetence by Garlic: Role in Bladder Cancer and other Malignancies, Journal of Nutrition, vol. 131, no. 13s, 2001, pages 1067s-1070s, it is thus demonstrated that the anti-tumoral activity of garlic is at least in part connected to an immune stimulation: increase phagocytosis, increase of the tiller cells (NK) and immunoproliferation of the lymphocytes are the principal observed phenomena.

The antiseptic properties of polyphenols are moreover well known.

More precisely again, the sulfurated antibacterial agents derived from allicin are extracts of plants of the Allium genus, and more precisely varieties of Allium sativum and Allium cepa.

Garlic, employed since antiquity as an antiseptic, has an antibacterial and antifungal activity which has been shown in vitro. These properties have been known for a long time by Pasteur, and garlic has been used empirically all over the world to treat microbial infections.

The plants of the garlic family, and more particularly the variety Allium cepa, show a wide antibiotic spectrum for gram positive and gram negative bacteria. Garlic juice is itself efficacious against most pathogenic bacteria of the digestive tract, even on the strains resistant to antibiotics.

According to the invention, the component or components aiming to reduce the adhesion of germs to the wall are anthocyanosids, organic acids and polyphenol derivatives extracted from at least one member of the genus Ericaceae, Saxifragaceae, Vitaceae and Caprifoliaceae and/or fruit fructose.

According to one possibility, said components can be extracted from the family Ericaceae, and more precisely from the genus Vaccinium and the species Vaccinium myrtillus, Vaccinium corymbosum, and Vaccinium macrocarpon.

Taking as an example Vaccinium macrocarpon, this septentrional species increases spontaneously in the eastern part of North America, from the Carolinas to Canada. It produces small dark red fruit, widely consumed in nature as well as in the form of juice. The beneficial bacteriostatic effect of the fruit juice in the treatment of urinary infections is confirmed by very ancient use. It seems that its action is connected to an inhibition of the adherence of the bacteria to the mucosa. This has been demonstrated in the case of the adhesion of Escherichia coli to the uroepithelial cells.

The patent WO 99/12541 proposes in this connection certain purified extracts of proanthocyanidines of plants of the family Ericaceae especially, and of the genus Vaccinium in particular, which are capable of selectively inhibiting the adherence of the bacteria E. Coli of the P type, involved in renal infections, and not that of the bacteria E. Coli of type 1, involved in infections of the bladder.

The compositions included in such extracts are adapted for the treatment of infections of the urinary tract.

According to certain studies, this plant would have the virtue of prevention of recurrence of urinary infections, and thus would be an interesting alternative to the other therapies seeking to space apart the recurrences.

Alternatively, according to the invention, these components can be extracted from the family Saxifragaceae, and more particularly of the species Ribes nigrum.

As still another modification, they can be extracted from the family Vitaceae, and more precisely from the species Vitis vinifera.

Again alternatively, they can be extracted from the genus Caprifoliaceae, and more precisely from Sambucus nigra.

Moreover, according to another of its principal characteristics, the composition of the invention contains polyunsaturated fatty acids of the family n-3, aimed at improving the defense mechanisms of the mucosa.

The precursor of the family of polyunsaturated n-3 fatty acids is linoleic acid, which is found in the tissues of green plants and in certain oils of vegetable origin. In the organism, linoleic acid is converted into eicosapentaenoic acids (EPA) and docosahexaenoic acids (DHA).

The food of developed countries is about 10 times richer in linolenic acid, precursor of the family of polyunsaturated n-6 fatty acids, than in linoleic acid, and the metabolism of the first accordingly dominates in the human body.

Thus, our cells contain only small quantities of EPA and DHA following on the alimentary desequilibria mentioned. However, it has been determined in a surprising manner that certain populations which consume large quantities of fish and fish oil, which are rich in EPA and in DHA, have a very low incidence of inflammatory and autoimmune maladies. This suggests in particular that these fatty acids insert themselves in place of arachidonic acid, which is a precursor of prostaglandins and of leukotrienes which have strong pro-inflammatory properties. This insertion thus permits inhibiting in a competitive manner the conversion of the arachidonic acid into prostaglandines. This is accompanied by a decrease in the production by the inflammatory cells of the inflammation mediators such as the prostaglandines and the leukotrienes. EPA and DHA also act on certain mechanisms of the immune system and thus reestablish its equilibrium, thereby avoiding the substantial de-granulation of the mastocytes and the damage created in the urothelium.

Thus, in the invention, the polyunsaturated fatty acid is selected from among eicosapentaenoic acid (EPA) and docosahexaenoic acid (DHA).

Again more particularly, eicosapentaenoic acid being present in fish oil, the composition for the prevention of infections of the urinary system according to the invention contains such a fish oil.

According to a possibility resulting from the possible choices such as mentioned at the outset, the composition of the invention is characterized in that it contains:
Vaccinium macrocarpon;
Allium sativum; and
fish oil.

More precisely, it is packaged so as to contain:
100 to 1000 mg of Vaccinium macrocarpon extract;
10 to 1000 mg of Allium sativum extract; and
500 to 2000 mg of fish oil.

Of course, the choice of packaging or of the components of the composition of the invention, as well as the relative weights of said components, depend on the selection of the components carried out on the basis of the indications given in the present description. Thus, each one of Vaccinium macrocarpon, of allium sativum and of fish oil can be replaced by equivalent components, mentioned at the outset according to a dosage which is nevertheless not that which has been given of the three components and which forms a part of the know-how of those skilled in the art.

1-14. (canceled)

15. Composition for the prevention of infections of the urinary system, characterized in that:
for the suppression of pathogenic germs and/or the reduction of their noxiousness, it comprises:
sulfurated compounds derived from allicin aiming to exert an antibacterial effect on the germs present in the urinary tract and the gastrointestinal tract, so as to decrease the colonization by these latter of the urinary tract, and
components selected from the group constituted by anthocyanosids and polyphenolic derivatives, extracted from at least one of the families Ericaceae, Saxifragaceae, Vitaceae and Caprifoliaceae and/or fruit fructose, as components which air to reduce the adhesion of the germs to the wall,
and in that
for the reinforcement of the integrity of the wall of the urinary tract, it comprises polyunsaturated fatty acids of the n-3 family permitting decreasing the inflammation of this latter and rebalancing the immune system.

16. Composition for the prevention of infections of the urinary system according to claim 15, characterized in that the sulfurated compounds derived from allicin are extracts of plants of the genus Allium and more precisely the species Allium sativum and Allium cepa.

17. Composition for the prevention of infections of the urinary system according to claim 15, characterized in that said components to reduce the adhesion of the germs to the wall are extracts of the family Ericaceae and more precisely of the genus Vaccinium and of the species Vaccinium myrtillus, Vaccinium corymbosum and Vaccinium macrocarpon.

18. Composition for the prevention of infections of the urinary system according to claim 15, characterized in that said components to reduce the adhesion of the germs to the wall are extracts of the family Saxifragaceae and more particularly of the species Ribes nigrum.

19. Composition for the prevention of infections of the urinary system according to claim 15, characterized in that said components to reduce the adhesion of the germs to the wall are extracts of the family Vitaceae and more precisely of the species Vitis vinifera.

20. Composition for the prevention of infections of the urinary system according to claim 15, characterized in that said components to reduce the adhesion of the germs to the wall are extracts of the genus Caprifoliaceae and more precisely of Sambucus nigra.

21. Composition for the prevention of infections of the urinary system according to claim 15, characterized in that the polyunsaturated fatty acid is selected from among eicosapentaenoic acid (EPA) and docosahexaenoic acid (DHA).

22. Composition for the prevention of infections of the urinary system according to claim 15, characterized in that it contains fish oil as a substance comprising at least one polyunsaturated fatty acid.

23. Composition for the prevention of infections of the urinary system according to claim 15, characterized in that it contains:
Vaccinium macrocarpon;
Allium sativum; and
fish oil.

24. Composition for the prevention of infections of the urinary system according to claim 23, characterized in that it is packaged so as to contain:
100 to 1000 mg of Vaccinium macrocarpon extract;
10 to 1000 mg of Allium sativum extract; and
500 to 2000 mg of fish oil.
